(19) United States
(12) Patent Application Publication
(10) Pub. No.: US 2026/0269079 A1
(43) Pub. Date: Sep. 10, 2026

(54) POLYGENIC RISK SCORE VISUALIZATION METHOD AND APPARATUS

(71) Applicant: SEOUL NATIONAL UNIVERSITY R&DB FOUNDATION, Seoul (KR)

(72) Inventors: Seunggeun LEE, Seoul (KR); Na Yeon KIM, Seongnam-si Gyeonggi-do (KR)

(21) Appl. No.: 19/669,301

(22) Filed: May 6, 2026

Related U.S. Application Data
(63) Continuation of application No. PCT/KR2024/017452, filed on Nov. 7, 2024.

(30) Foreign Application Priority Data
Nov. 9, 2023 (KR) 10-2023-0154076
Nov. 6, 2024 (KR) 10-2024-0155884

(51) Int. Cl. G16H 50/30; G16B 20/20; G16B 45/00; G16H 15/00; G16H 50/70

(52) U.S. Cl. CPC G16H 50/30; G16B 20/20; G16B 45/00; G16H 15/00; G16H 50/70

(57) ABSTRACT

An embodiment relates to a polygenic risk score visualization method. The method includes confirming whether input genotype data matches prestored genetic variation risk data and mapping genes and genetic variants based on the genotype data, calculating a polygenic risk score, attributed values, and variant contribution scores for the genetic variants based on a plurality of preset algorithms, and providing visualization by visualizing gene contribution scores for each gene at a population level, attributed values for each gene at an individual level, and variant contribution scores for each gene at an individual level based on the attributed values and the variant contribution scores.

### CROSS-REFERENCE TO RELATED APPLICATION

This application is based on and claims priority under 35 U.S.C. § 119 to Korean Patent Application No. 10-2024-0155884, filed on Nov. 6, 2024, No. 10-2023-0154076, filed on Nov. 9, 2023 in the Korean Intellectual Property Office, and continuation of International Application No. PCT/KR2024/017452 filed on Nov. 7, 2024 the disclosure of which is incorporated by reference herein in its entirety.

### BACKGROUND

### Technical Field

The present invention relates to a polygenic risk score visualization method and apparatus, and more particularly, to a method and apparatus for performing mapping of genetic variants to genes based on genotype data, calculating a polygenic risk score, attributed values and contribution scores for the gene and the genetic variants according to a plurality of preset algorithms, and providing visualization data therefor.

### Background Art

Disease risk prediction is an essential part of preventive medicine and guides clinical management. In general, disease risk prediction includes risk factors such as age, sex, family history, and lifestyle.

Recently, many studies have confirmed that predictive performance is improved when a disease risk prediction model is constructed using a polygenic risk score (PRS) obtained by aggregating genomic information.

The simplest and most common form of the polygenic risk score is a sum obtained by multiplying the number of risk genetic variants (Single Nucleotide Polymorphisms, SNPs) possessed by an individual by β (weight) values of the genetic variants obtained from Genome Wide Association Study (GWAS) summary statistics, and is represented as a single number proportional to the risk of a disease.

However, since the polygenic risk score is generally composed of a combination of hundreds to tens of millions of genetic variants, there is a problem in that it is difficult to directly determine which genetic factor increases the risk of the disease.

In addition, existing methods for constructing polygenic risk scores, such as LDpred, PRS-CS, and LassoSum, have only attempted to improve the predictive power of the polygenic risk score, and studies on methods for interpreting the polygenic risk score have not been conducted.

Accordingly, there is a need for a method capable of providing an explanation for the polygenic risk score.

### SUMMARY

### Problem to be Solved

The present invention is intended to solve the above-described problems of the conventional technology, and relates to a method and apparatus for performing mapping of genetic variants to genes based on genotype data, calculating a polygenic risk score, attributed values, and a contribution scores for the gene and the genetic variants according to a plurality of preset algorithms, and providing visualization data therefor.

The technical problems to be achieved by the present invention are not limited to the above-described technical problems, and other technical problems of the present invention may be derived from the following description.

### Means for Solving the Problem

As a technical means for solving the above-described technical problem, an embodiment according to a first aspect of the present disclosure provides a polygenic risk score visualization method.

The method includes confirming whether input genotype data matches prestored genetic variation risk data, mapping genes and genetic variants based on the genotype data, calculating a polygenic risk score, attributed values, and variant contribution scores for the genetic variants based on a plurality of preset algorithms, and providing visualization by visualizing gene contribution scores for each gene at a population level, attributed values of the gene and variant contribution scores at an individual level.

As a technical means for solving the above-described technical problem, an embodiment according to a second aspect of the present disclosure provides a polygenic risk score visualization apparatus. The apparatus includes a communication module, at least one processor, and a memory electrically connected to the processor and storing at least one code executed by the processor, wherein the memory stores code that, when executed by the processor, causes the processor to confirm whether input genotype data matches prestored genetic variation risk data, map genes and genetic variants based on the genotype data, calculate a polygenic risk score, attributed values, and variant contribution scores for the genetic variants based on a plurality of preset algorithms, and provide visualization by visualizing gene contribution scores for each gene at a population level, attributed values for each gene at an individual level, and variant contribution scores for each gene at an individual level based on the attributed values and the variant contribution scores.

### Effects of the Invention

According to the present invention, the method enables identification of which genetic factors may cause the manifestation of a disease in an individual.

In addition, according to the present invention, convenience may be improved since existing methods for constructing a polygenic risk score (PRS) may be utilized as they are.

In addition, according to the present invention, explanation of genetic factors may facilitate communication between clinicians and patients.

In addition, according to the present invention, drug prescription or treatment targeting genetic factors becomes possible, thereby enabling provision of personalized medical services.

In addition, according to the present invention, the method may assist in developing new drugs for preventing or treating diseases.

The effects of the present invention are not limited to the above-described effects, and include all effects that can be understood from the following description.

### BRIEF DESCRIPTION OF THE DRAWINGS

FIG. 1 is a diagram illustrating a server according to an embodiment of the present invention and a terminal communicatively connected thereto.

FIG. 2 is a diagram illustrating a detailed configuration of the server shown in FIG. 1.

FIG. 3 is a diagram illustrating an example of preprocessing.

FIGS. 4 to 6 are diagrams illustrating examples for calculating a polygenic risk score, attributed values, and variant contribution scores.

FIGS. 7 to 11 are diagrams illustrating examples of visualization.

FIG. 12 is a flowchart illustrating a sequence of a polygenic risk score visualization method according to another embodiment of the present invention.

FIGS. 13 and 14 are flowcharts illustrating sequences of detailed steps of the polygenic risk score visualization method shown in FIG. 12.

### DETAILED DESCRIPTION

Hereinafter, the present disclosure will be described in detail with reference to the accompanying drawings. However, the present disclosure may be implemented in various different forms and is not limited to the embodiments described herein. In addition, the accompanying drawings are provided only to facilitate understanding of the embodiments disclosed in the present specification, and the technical idea disclosed in the present specification is not limited by the accompanying drawings. All terms including technical and scientific terms used herein should be interpreted as having meanings commonly understood by a person having ordinary skill in the art to which the present disclosure pertains. Terms defined in advance should be interpreted as additionally having meanings consistent with the related technical documents and the presently disclosed contents, and unless defined otherwise, should not be interpreted in an excessively ideal or overly restrictive sense.

In the drawings, portions irrelevant to the description have been omitted in order to clearly explain the present disclosure, and the sizes, shapes, and forms of respective components illustrated in the drawings may be variously modified. Throughout the specification, the same or similar reference numerals are assigned to the same or similar components.

Suffixes such as “module” and “unit” for components used in the following description are provided or used interchangeably only for convenience in preparing the specification and do not in themselves have distinct meanings or roles. In addition, in describing the embodiments disclosed in the present specification, when it is determined that a detailed description of related well-known technology may obscure the gist of the embodiments disclosed in the present specification, the detailed description thereof will be omitted.

Throughout the specification, when a part is described as being “connected (coupled, contacted, or combined)” to another part, this includes not only a case in which the part is “directly connected (coupled, contacted, or combined)” but also a case in which the part is “indirectly connected (coupled, contacted, or combined)” with another member interposed therebetween. In addition, when a part is described as “including (comprising or being provided with)” a certain component, this means that the part may further include other components unless specifically stated otherwise.

In the present specification, terms indicating ordinal numbers such as first and second are used only for the purpose of distinguishing one component from another component and do not limit the order or relationship of the components. For example, a first component of the present disclosure may be referred to as a second component, and similarly, a second component may also be referred to as a first component. Singular expressions used herein should be interpreted as including plural expressions unless clearly indicated otherwise.

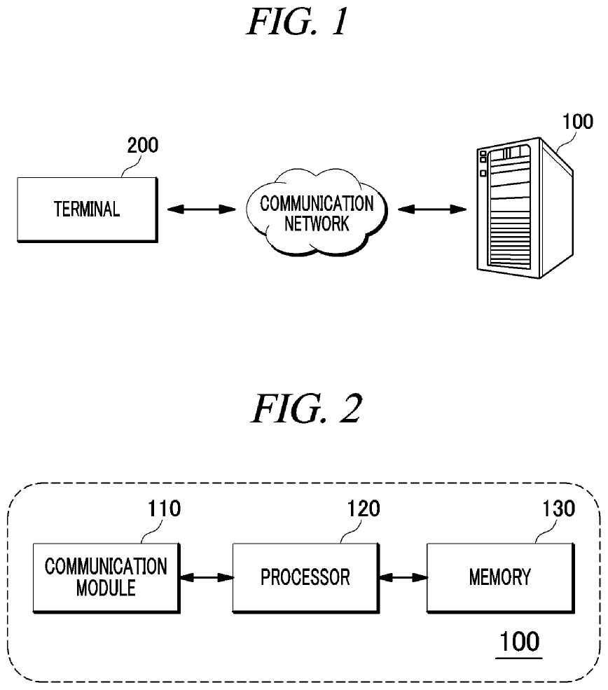

FIG. 1 is a diagram illustrating a server according to an embodiment of the present invention and a terminal communicatively connected thereto.

Referring to FIG. 1, a server (100) may be communicatively connected to a terminal (200) through a preset communication network.

The server (100) stores code that causes confirmation of whether input genotype data matches prestored genetic variation risk data and mapping of genes and genetic variants based on the genotype data.

Here, the genotype data may be in a pLink format. For example, a file in the pLink format may include a bed file storing genotype information in a binary format, a bim file storing information on genetic variants, and a fam file storing family information of individuals.

In addition, the genotype data may include information on at least one of a gene, a genetic variation identifier, a position, A1, A2, a family identifier, an individual identifier, sex, and a phenotype.

The server (100) calculates a polygenic risk score, attributed values, and variant contribution scores for genetic variants based on a plurality of preset algorithms.

Here, the polygenic risk score may be a weighted sum of the number of risk genetic variants (Single Nucleotide Polymorphisms, SNPs) possessed by an individual, the attribute value may be a factor used to determine which gene increases the risk of a disease for a specific population or for an individual, and the variant contribution score may be a score for identifying genetic variants that increase each gene contribution score.

The server (100) provides visualization based on the attributed values and the variant contribution scores by visualizing gene contribution scores for each gene at a population level, attributed values for each gene at an individual level, and variant contribution scores for each gene at an individual level. For example, the server (100) may visualize the gene contribution scores for each gene at a population level, the attributed values for each gene at an individual level, and the variant contribution scores for each gene at an individual level in the form of graphs.

The terminal (200) may transmit genotype data to the server (100) and may receive visualization information from the server (100).

The terminal (200) may refer to any type of handheld-based wireless communication device such as a notebook, desktop, or laptop equipped with a web browser, or a wireless communication device ensuring portability and mobility such as a smartphone or a tablet PC.

FIG. 2 is a diagram illustrating a detailed configuration of the server shown in FIG. 1.

Referring to FIG. 2, the server (100) may include a communication module (110), a processor (120), and a memory (130).

The communication module (110) may include a device including hardware and software required for transmitting and receiving signals such as control signals or data signals through wired or wireless connections with other network devices.

The communication module (110) may receive genomic data from a terminal and may transmit visualization information to the terminal.

The processor (120) may include various types of devices for controlling and processing data.

The processor (120) may refer to a data processing device embedded in hardware and having a physically structured circuit for performing functions expressed as code or instructions included in a program.

In one example, the processor (120) may be implemented in the form of a microprocessor, a central processing unit (CPU), a processor core, a multiprocessor, an application-specific integrated circuit (ASIC), or a field programmable gate array (FPGA), but the scope of the present invention is not limited thereto.

The processor (120) performs operations according to code stored in the memory (130).

The memory (130) may store at least one of information and data input through the communication module (110), information and data required for functions performed by the processor (120), and data generated according to execution of the processor (120).

The memory (130) should be interpreted as collectively referring to a non-volatile storage device that continuously retains stored information even when power is not supplied and a volatile storage device that requires power to retain stored information. The memory (130) may include, in addition to a volatile storage device requiring power to retain stored information, cloud storage, a solid-state drive (SSD), magnetic storage media, or flash storage media, but the scope of the present invention is not limited thereto.

The memory (130) is electrically connected to the processor (120), and stores at least one code executed by the processor (120). The memory (130) stores code that, when executed by the processor (120), causes the processor (120) to perform the following functions and procedures.

The memory (130) stores code that causes confirmation of whether input genotype data matches prestored genetic variation risk data and mapping of genes and genetic variants based on the genotype data.

The memory (130) may store code that causes a sign of an effect size vector of a genetic variation to be changed according to whether the input genotype data matches the prestored genetic variation risk data. Here, the genetic variation risk data may be a file of a polygenic risk score including the effect size vector of the genetic variation. The genetic variation risk data may be in the form of a file obtained from a polygenic risk score construction method such as lassosum or PRS-CS and may include information on a gene, a genetic variation identifier, a position, A1, A2, and an effect size. The effect size may be the same as a weight value of the genetic variation.

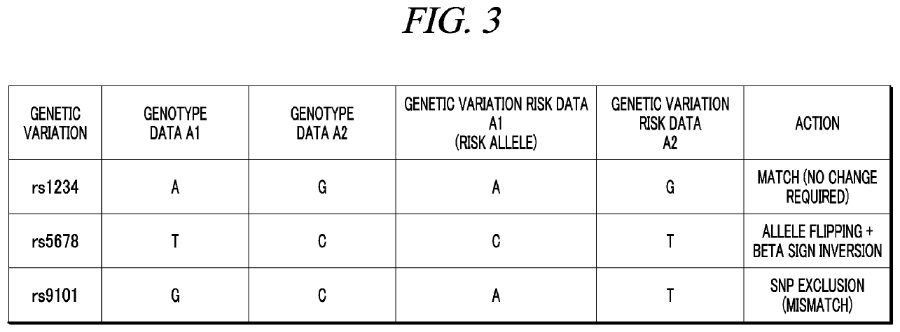

For example, the memory (130) may store code that causes comparison of A1 and A2 between the genotype data and the genetic variation risk data in order to determine whether they match. The memory (130) may store code that causes maintenance of a current state when the matching result indicates that the values match, exclusion of the corresponding genetic variation when the matching result indicates that the values do not match, and change of the sign of the effect size vector when the matching result indicates that the values match but do not correspond. Detailed description related thereto will be described later with reference to FIG. 3.

The memory (130) may store code that causes genetic variants to be mapped to genes based on first to third methods.

The memory (130) may store code that causes primary mapping of genes and genetic variants to be performed based on a distance between genes and genetic variants.

For example, since genetic variants located close to gene A may affect gene A, genetic variants located within a preset distance from gene A may be mapped to gene A.

The memory (130) may store code that causes secondary mapping of genes and genetic variants to be performed based on relationships between genes and genetic variants for which primary mapping has not been performed.

For example, the memory (130) may store code that causes genes and genetic variants to be mapped based on a Combining SNP-to-Gene (CS2G) mapping method. The Combining SNP-to-Gene (CS2G) mapping method may be a technique for identifying relationships between genetic variants and genes based on Genome-Wide Association Study (GWAS) results, gene expression information, and interaction networks.

The memory (130) may store code that causes extraction of genetic variants to be mapped based on connectivity between genetic variants and diseases for which secondary mapping has not been performed, and performance of tertiary mapping by setting a corresponding region within a preset data size based on the genetic variants to be mapped.

As an example, the memory (130) may store code that causes mapping of regions based on genetic variants in order of genetic variants having low p-values among genetic variants in a file when GWAS summary statistics are given, based on a GWAS p-value/SNP heritability based mapping method.

Here, the p-value represents a statistically associated degree, and a very low p-value may indicate that a genetic variation is significantly associated with a specific trait.

For example, the memory (130) may store code that causes analysis based on significant genetic variants having low p-values in order to determine which gene or gene region each genetic variation belongs to. Through this, genes strongly associated with a specific trait or disease may be estimated.

As another example, the memory (130) may store code that causes mapping of regions with a preset data size based on genetic variants in order of genetic variants having high heritability by calculating heritability from genotype data, based on a GWAS p-value/SNP heritability based mapping method.

For example, the memory (130) may store code that causes evaluation of contributions of individual genetic variants to traits and calculation of total genetic effects of a plurality of genetic variants on the traits.

The memory (130) may store code that causes grouping of genes into a single group when genetic variants matching a preset probability or higher through mapping are identical. For example, when genetic variants mapped to gene A are a, b, and c, genetic variants mapped to gene B are b, c, and d, and the preset probability is 2/3, gene A and gene B may be grouped into a single group based on code stored in the memory (130).

The memory (130) stores code that causes calculation of a polygenic risk score, attributed values, and variant contribution scores for genetic variants based on a plurality of preset algorithms.

The memory (130) may store code that causes calculation of a polygenic risk score based on a preset first algorithm.

The preset first algorithm may be expressed as the following Equation 1.

PRS=β× [Equation 1]

Here, β may be an effect size vector of genetic variants, and X may be a genotype matrix.

The memory (130) may store code that causes calculation of a gene contribution score based on the polygenic risk score. Here, the gene contribution score may be a weighted sum of genetic variants mapped to a gene and the number of genetic variants possessed by an individual.

The gene contribution score may be calculated based on the following Equation 2.

CS_{(≠i)}=β_{(≠i)} X_{(≠i)} [Equation 2]

Here, β_{(≠i)} may be a weight vector of genetic variants included in gene i, and X_{(≠i)} may be a genotype matrix of genetic variants included in gene i.

The memory (130) may store code that causes calculation of attributed values based on the polygenic risk score according to a preset second algorithm. Here, the attribute value may be used to determine which gene increases the risk of a disease for a specific population or for an individual.

The preset second algorithm may be calculated based on the following Equation 3.

A_{(≠i)}=CS_{(≠i)}−(CS_{(≠i)})⁻ [Equation 3]

Here, (CS_{(≠i)})⁻ may be an average of gene contribution scores of a population.

The memory (130) may store code that causes selection of a risk gene within a specific population as a gene having the largest variance of gene contribution scores within a region, E[(A_{(≠i)})²].

In addition, the memory (130) may store code that causes selection of a risk gene of an individual as a gene having the largest attribute value A_{(≠i)} within the region.

The memory (130) may store code that causes calculation of variant contribution scores based on a preset third algorithm.

The third algorithm may be calculated based on the following Equation 4.

CS(snpₖ)=β×x [Equation 4]

Here, β may be a weight vector of genetic variants, and x may be the number of risk genetic variants. For example, x may be one of 0, 1, and 2.

The memory (130) stores code that causes visualization to be provided by visualizing gene contribution scores for each gene at a population level, attributed values for each gene at an individual level, and variant contribution scores for each gene at an individual level based on the attributed values and the variant contribution scores.

For example, the memory (130) may store code that causes visualization to be provided by visualizing gene contribution scores for each gene at a population level, attributed values for each gene at an individual level, and variant contribution scores for each gene at an individual level based on the attributed values and the variant contribution scores at a population level and an individual level.

The memory (130) may store code that causes visualization using a Manhattan plot to represent important genes at a population level and an individual level, and visualization using a locuszoom-like plot to represent important genetic variants at an individual level.

FIG. 3 is a diagram illustrating an example of preprocessing.

Referring to FIG. 3, the polygenic risk score visualization apparatus may confirm whether risk genetic variants between genotype data and genetic variation risk data match.

For the rs1234 genetic variation, when A1 of the genotype data is A, A2 of the genotype data is G, A1 of the genetic variation risk data is A, and A2 of the genetic variation risk data is G, the polygenic risk score visualization apparatus may determine that the genotype data and the genetic variation risk data match since A1 of the genotype data matches A1 of the genetic variation risk data and A2 of the genotype data matches A2 of the genetic variation risk data.

For the rs5679 genetic variation, when A1 of the genotype data is T, A2 of the genotype data is C, A1 of the genetic variation risk data is C, and A2 of the genetic variation risk data is T, the polygenic risk score visualization apparatus may perform allele flipping and invert the sign of the effect size vector since A1 of the genotype data matches A2 of the genetic variation risk data and A2 of the genotype data matches A1 of the genetic variation risk data.

For the rs5679 genetic variation, when A1 of the genotype data is G, A2 of the genotype data is C, A1 of the genetic variation risk data is A, and A2 of the genetic variation risk data is T, the rs5679 genetic variation may be excluded since A1 of the genotype data does not match A2 of the genetic variation risk data.

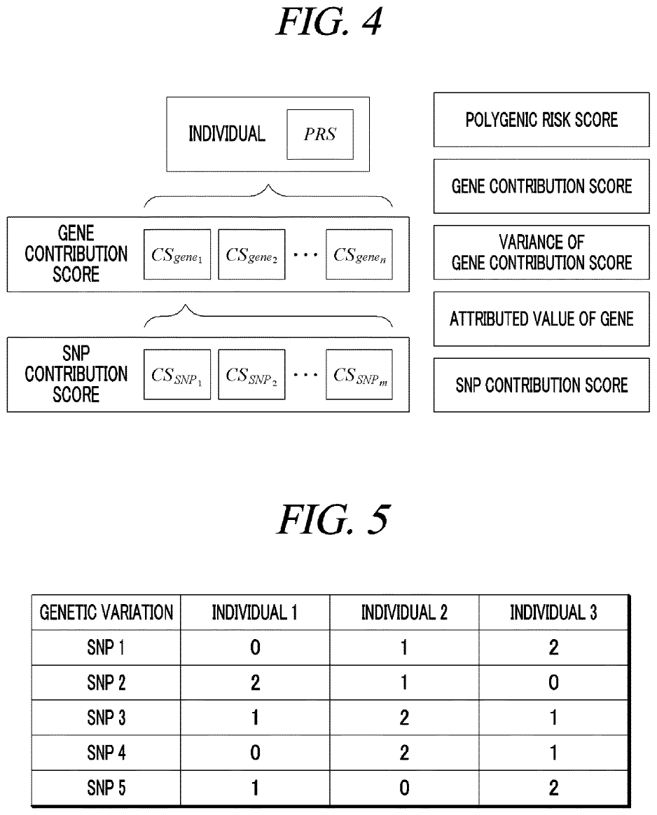

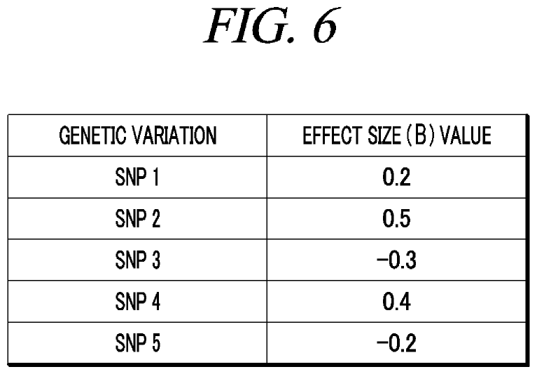

FIGS. 4 to 6 are diagrams illustrating an example for calculating a polygenic risk score, attributed values, and variant contribution scores. More specifically, FIG. 4 is a diagram illustrating a process for calculating a polygenic risk score, attributed values of genes, and variant contribution scores, FIG. 5 is a diagram illustrating an example of a genotype matrix, and FIG. 6 is a diagram illustrating an example of effect sizes of genetic variants.

Referring to FIGS. 4 to 6, the polygenic risk score visualization apparatus may calculate a polygenic risk score on an individual basis.

The polygenic risk score visualization apparatus may calculate a gene contribution score after calculating the polygenic risk score on an individual basis.

The polygenic risk score visualization apparatus may calculate a variance of gene contribution scores after calculating the gene contribution scores.

The polygenic risk score visualization apparatus may calculate an attribute value of a gene after calculating the variance of the gene contribution scores.

The polygenic risk score visualization apparatus may calculate a SNP contribution score.

An example of the genotype matrix required for calculating the polygenic risk score and the gene contribution score may be as illustrated in FIG. 5.

For SNP 1, SNP 2, SNP 3, SNP 4, and SNP 5, the genotype matrix of Individual 1 may be 0, 2, 1, 0, and 1, respectively.

For SNP 1, SNP 2, SNP 3, SNP 4, and SNP 5, the genotype matrix of Individual 2 may be 1, 1, 2, 2, and 0, respectively.

For SNP 1, SNP 2, SNP 3, SNP 4, and SNP 5, the genotype matrix of Individual 3 may be 2, 0, 1, 1, and 2, respectively.

An example of effect sizes of genetic variants required for calculating the polygenic risk score and the gene contribution score may be as illustrated in FIG. 6.

Effect size (Beta) values for SNP 1, SNP 2, SNP 3, SNP 4, and SNP 5 may be 0.2, 0.5, −0.3, 0.4, and −0.2, respectively.

The polygenic risk score visualization apparatus may calculate polygenic risk scores for each individual.

The polygenic risk score of Individual 1 may be calculated as 0×0.2+2×0.5+1×(−0.3)+0×0.4+1×(−0.2)=0.5.

The polygenic risk score of Individual 2 may be calculated as 1×0.2+1×0.5+2×(−0.3)+2×0.4+0×(−0.2)=0.9.

The polygenic risk score of Individual 3 may be calculated as 2×0.2+0×0.5+1×(−0.3)+1×0.4+2×(−0.2)=0.1.

Assuming that SNP 1, SNP 2, and SNP 3 are mapped to Gene 1 through gene mapping, the polygenic risk score visualization apparatus may calculate gene contribution scores for Individual 1, Individual 2, and Individual 3.

The gene contribution score of Individual 1 may be calculated as 0×0.2+2×0.5+1×(−0.3)=0.7.

The gene contribution score of Individual 2 may be calculated as 1×0.2+1×0.5+2×(−0.3)=0.1.

The gene contribution score of Individual 3 may be calculated as 2×0.2+0×0.5+1×(−0.3)=0.1.

The polygenic risk score visualization apparatus may calculate attributed values of genes and a variance of the attributed values.

An average value related to the gene contribution scores may be calculated as (0.7+0.1+0.1)/3=0.3.

The attributed value of the gene of Individual 1 may be calculated as 0.7-0.3=0.4.

The attributed value of the gene of Individual 2 may be calculated as 0.1-0.3=−0.2.

The attributed value of the gene of Individual 3 may be calculated as 0.1-0.3=−0.2.

The polygenic risk score visualization apparatus may calculate the variance of the gene contribution scores.

The variance of the gene contribution scores may be calculated based on the following Equation 5.

Var(CS_{(≠i)})=E[(CS_{(≠i)}−(CS_{(≠i)})⁻)²]=E[(A_{(≠i)})²] [Equation 5]

Based on Equation 5, the polygenic risk score visualization apparatus may calculate the variance of the gene contribution score of Gene 1 as 0.08, the variance of the gene contribution score of Gene 2 as 0.04, and the variance of the gene contribution score of Gene 3 as 0.04.

The polygenic risk score visualization apparatus may determine Gene 1 as the most risky gene based on the variance of the gene contribution scores.

The polygenic risk score visualization apparatus may calculate SNP contribution scores.

The SNP 1 contribution score may be calculated as 0×0.2=0.

The SNP 2 contribution score may be calculated as 2×0.5=1.0.

The SNP 3 contribution score may be calculated as 1×(−0.3)=−0.3.

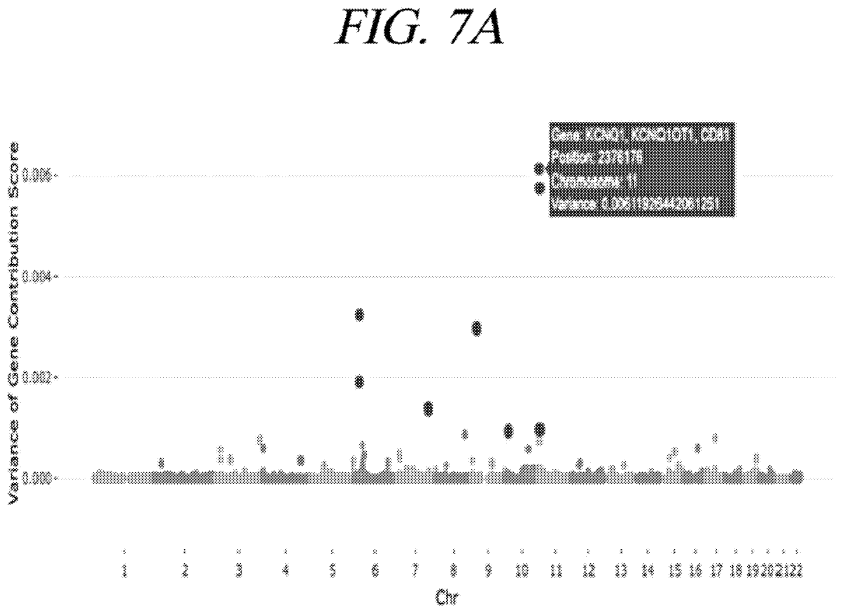

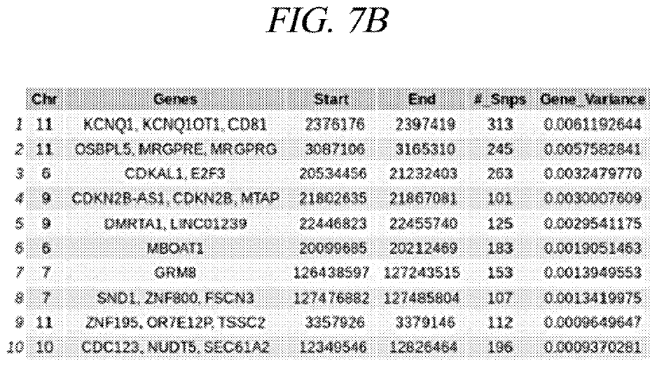

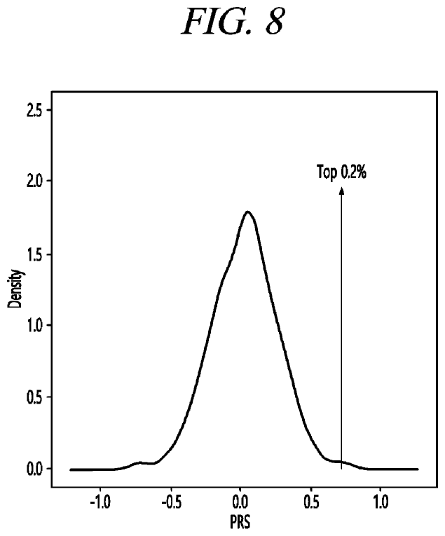

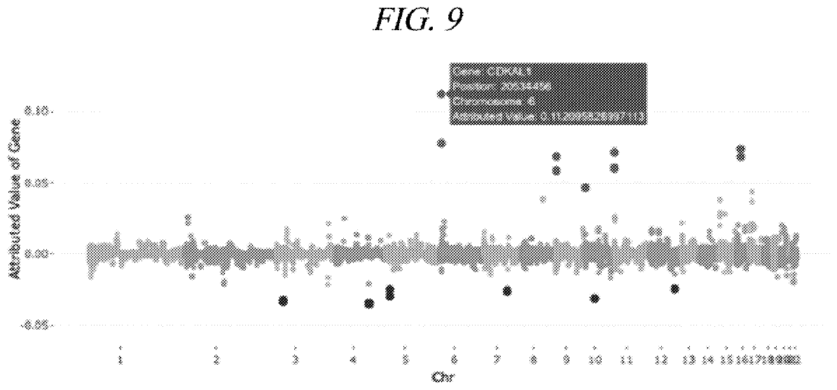

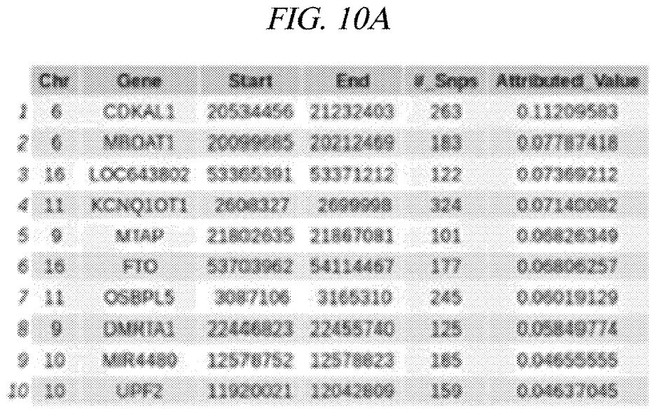

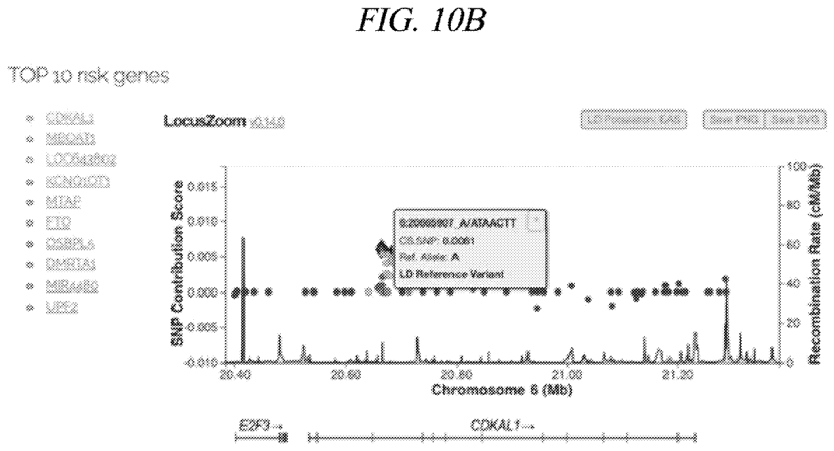

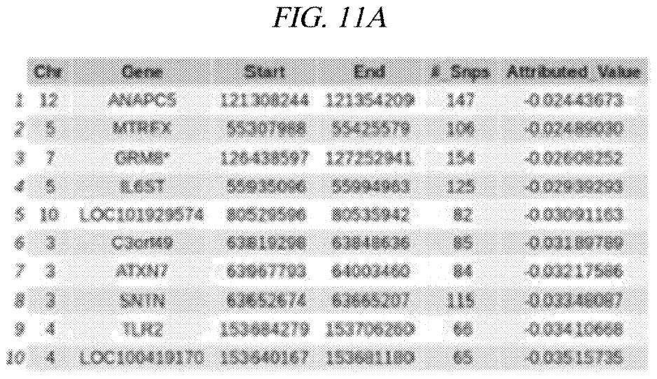

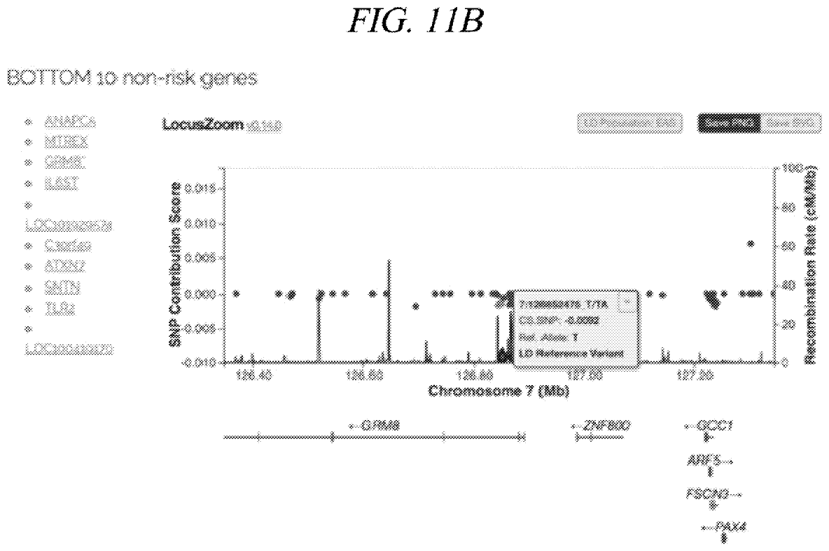

FIGS. 7 to 11 are diagrams illustrating examples of visualization. More specifically, FIGS. 7A and 7B are diagrams illustrating examples of visualization at a population level, FIG. 8 is a diagram illustrating an example of a density of a polygenic risk score, FIG. 9 is a diagram illustrating an example of attributed values at an individual level, FIGS. 10A and 10B are diagrams illustrating examples of upper variant contribution scores at an individual level, and FIGS. 11A and 11B are diagrams illustrating examples of lower variant contribution scores at an individual level.

Referring to FIGS. 7 to 11, the polygenic risk score visualization apparatus may provide a Manhattan plot at a population level visualizing importance of risk genes associated with type 2 diabetes and a list of the top 10 risk genes.

As shown in FIG. 7A, an x-axis of the Manhattan plot at the population level represents chromosome numbers and indicates on which chromosome each gene is located, and a y-axis represents a variance of gene contribution scores and may show variability of contributions of specific genes to a disease or trait.

Red dots in the Manhattan plot at the population level may represent the top 10 risk genes. Genes indicated in red may be genes exhibiting high risk in the population.

For example, in the Manhattan plot at the population level, genes KCNQ1, KCNQ1OT1, and CD81 located on chromosome 11 may be identified as risk genes, and genetic variants thereof may significantly influence the corresponding trait or disease.

As shown in FIG. 7A, the list of the top 10 risk genes at the population level may include information on chromosome numbers (Chromosome; Chr) in which the genes are located, gene names (Genes), start and end positions of the genes on the chromosomes (Start/End), numbers of genetic variants associated with the genes (#SNPs), and variances of gene contribution scores (Gene_Variance).

For example, genes KCNQ1, KCNQ1OT1, and CD81 are located on chromosome 11, and a gene contribution variance value thereof is 0.00611192644, which is the highest value, and thus these genes may be determined as risk genes.

As shown in FIG. 8, the polygenic risk score visualization apparatus may visualize gene contributions and a polygenic risk score (Polygenic Risk Score, PRS) of a specific individual (ID: HG00464) at an individual level.

A density graph may be provided. The density graph may represent a graph of polygenic risk scores of an entire population.

A red arrow in the density graph may indicate a position of a polygenic risk score of a specific individual (ID: HG00464) within the population.

For example, the polygenic risk score of the specific individual in the density graph may be 0.718082020126648, indicating that the individual belongs to the top 2%. The polygenic risk score visualization apparatus may determine that the specific individual has a relatively higher genetic risk for the corresponding disease than other individuals in the population.

As shown in FIG. 9, the polygenic risk score visualization apparatus may provide a gene-based Manhattan plot.

In the gene-based Manhattan plot, an x-axis may represent chromosomes on which genes are located, and a y-axis may represent attributed values of genes (Attributed Value of Gene), indicating a degree to which a specific gene contributes to a disease risk of an individual.

Red dots in the gene-based Manhattan plot may represent top risk genes in which an individual has a high risk. For example, the gene CDKAL1 located on chromosome 6 may appear as a top risk gene, and an attribute value of the gene may be high, such as 0.105060896911179.

Blue dots in the gene-based Manhattan plot may represent bottom risk genes in which an individual has relatively low risk.

As shown in FIG. 10A, the polygenic risk score visualization apparatus may provide a list of the top 10 risk genes of an individual. The list of the top 10 risk genes of the individual may include information on a chromosome number (Chromosome; Chr) in which each gene is located, a gene name (Genes), a start position and an end position of the gene on the chromosome (Start/End), a number of genetic variants associated with the gene (#SNPs), and an attribute value of the gene (Attributed Value).

Here, the attribute value of the gene may represent a value indicating how much the gene contributes to the disease risk of the individual. For example, a value of the gene CDKAL1 may be 0.105060896911179, and thus the gene may be determined to have a significant influence on the individual.

As shown in FIG. 10B, the polygenic risk score visualization apparatus may visualize variant contribution scores of the top 10 risk genes using a genetic variation-based locuszoom-like plot.

The genetic variation-based locuszoom-like plot may show how much each genetic variation contributes to a trait or disease. In the graph, a SNP contribution score and a recombination rate of genes may be displayed.

In the genetic variation-based locuszoom-like plot of the top 10 risk genes, a left x-axis may represent a portion where genes are located at megabase (Mb) positions on chromosome 6, a right x-axis may represent a recombination rate within a corresponding genetic variation region, where a higher value indicates a region where recombination occurs more frequently, and a y-axis may represent a degree to which each genetic variation contributes to a trait or disease.

Blue dots in the genetic variation-based locuszoom-like plot of the top 10 risk genes may represent visualized contributions of each genetic variation. For example, a genetic variation having the largest contribution in the graph may be 20:6963697_AT/A, and an AT allele may be interpreted as a major risk variant.

As shown in FIG. 11A, the polygenic risk score visualization apparatus may provide a list of the bottom 10 risk genes of an individual. The list of the bottom 10 risk genes of the individual may include information on a chromosome number (Chromosome; Chr) in which each gene is located, a gene name (Genes), a start position and an end position of the gene on the chromosome (Start/End), a number of genetic variants associated with the gene (#SNPs), and an attribute value of the gene (Attributed Value).

As shown in FIG. 11B, the polygenic risk score visualization apparatus may visualize variant contribution scores of the bottom 10 risk genes using a genetic variation-based locuszoom-like plot.

In the genetic variation-based locuszoom-like plot of the bottom 10 risk genes, a left x-axis may represent a portion where genes are located at megabase (Mb) positions on chromosome 7, a right x-axis may represent a recombination rate within a corresponding genetic variation region, and a y-axis may represent a degree to which each genetic variation contributes to a trait or disease.

FIGS. 8 to 11 described above illustrate a sample of an individual HG00464 from the 1000 Genomes Project, and the illustrated graph may represent polygenic risk scores of individuals within a population as a density plot. It may be confirmed that the individual belongs to the top 2% having a high genetic risk for type 2 diabetes.

The polygenic risk score visualization apparatus may provide a Manhattan plot and lists of the top 10 and bottom 10 risk genes of an individual, thereby enabling identification of which specific genes increase or decrease the polygenic risk score. For example, it may be confirmed that the gene CDKAL1 associated with type 2 diabetes has the highest attribute value of a gene. If the individual had an average attribute value, the polygenic risk score would be reduced by 0.112.

In addition, the polygenic risk score visualization apparatus may visualize contributions of polygenic risk scores within genes in detail through a locuszoom-like plot. For example, it may be confirmed that the individual HG00464 has 263 genetic variants in the gene CDKAL1, thereby increasing the polygenic risk score. Through such visualization, the polygenic risk score visualization apparatus may explain genetic risk of an individual at gene and genetic variation levels and may facilitate effective communication.

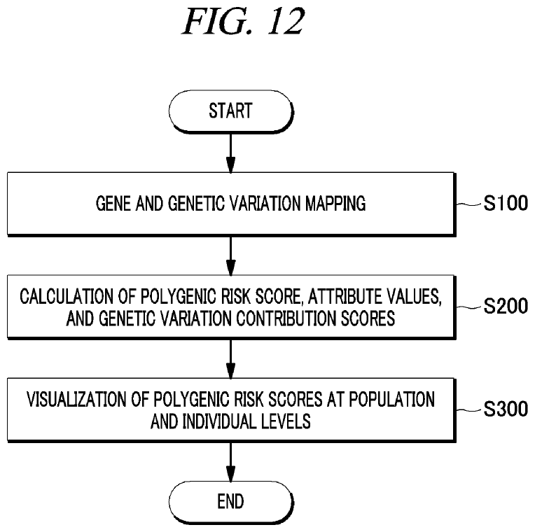

FIG. 12 is a flowchart illustrating a sequence of a polygenic risk score visualization method according to another embodiment of the present invention.

The polygenic risk score visualization method to be described below may be performed by the polygenic risk score visualization apparatus or the server (100) described above with reference to FIGS. 1 to 11. Accordingly, contents of the embodiments of the present disclosure described above with reference to FIGS. 1 to 11 may also be applied to the embodiments described below, and descriptions overlapping with those described above will be omitted. Steps described below are not necessarily performed sequentially, and an order of the steps may be variously arranged, and the steps may also be performed substantially simultaneously.

Referring to FIG. 12, the polygenic risk score visualization method includes a gene and genetic variation mapping step (S100), a polygenic risk score, attribute value, and variant contribution score calculation step (S200), and a population-level and individual-level polygenic risk score visualization step (S300).

The gene and genetic variation mapping step (S100) includes confirming whether input genotype data matches prestored genetic variation risk data and mapping genes and genetic variants based on the genotype data. The gene and genetic variation mapping step (S100) may include grouping genes into a single group when genetic variants matching a preset number or greater through mapping correspond to each other.

The polygenic risk score, attribute value, and variant contribution score calculation step (S200) is a step of calculating a polygenic risk score, attributed values, and variant contribution scores for genetic variants based on a plurality of preset algorithms.

The population-level and individual-level polygenic risk score visualization step (S300) is a step of providing visualization by visualizing gene contribution scores for each gene at a population level, attributed values for each gene at an individual level, and variant contribution scores for each gene at an individual level based on the attributed values and the variant contribution scores.

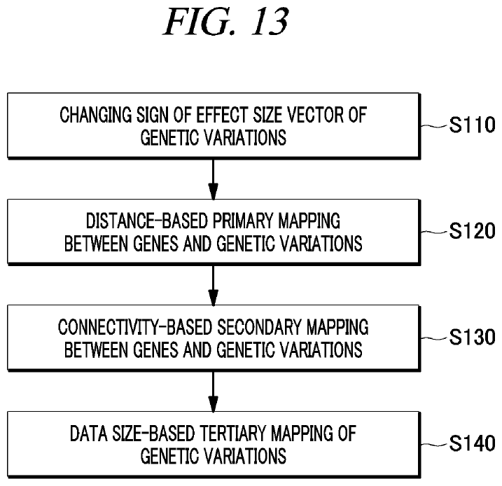

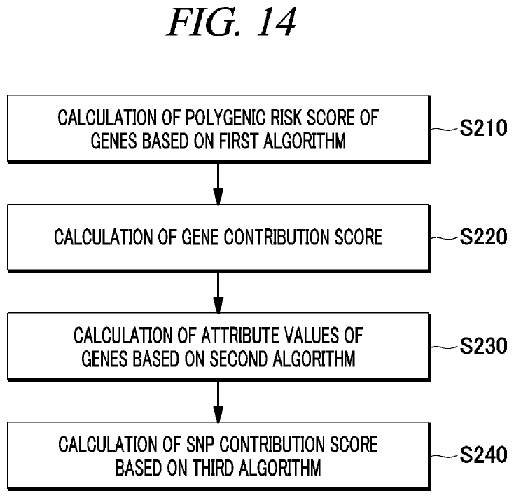

FIGS. 13 and 14 are flowcharts illustrating sequences of detailed steps of the polygenic risk score visualization method shown in FIG. 12.

Referring to FIG. 13, the polygenic risk score, attribute value, and variant contribution score calculation step (S200) may include a step (S110) of changing a sign of an effect size vector of genetic variants, a distance-based primary mapping step (S120) between genes and genetic variants, a connectivity-based secondary mapping step (S130) between genes and genetic variants, and a data size-based tertiary mapping step (S140) of genetic variants.

The step (S110) of changing the sign of the effect size vector of genetic variants may be a step of changing the sign of the effect size vector of genetic variants according to whether input genotype data matches prestored genetic variation risk data.

The distance-based primary mapping step (S120) between genes and genetic variants may be a step of performing primary mapping of genes and genetic variants based on a distance between the genes and the genetic variants.

The connectivity-based secondary mapping step (S130) between genes and genetic variants may be a step of performing secondary mapping of genes and genetic variants based on connectivity between genes and genetic variants for which primary mapping has not been performed.

The data size-based tertiary mapping step (S140) of genetic variants may be a step of extracting genetic variants to be mapped based on connectivity between genetic variants and diseases for which secondary mapping has not been performed and performing tertiary mapping by setting a corresponding region within a preset data size based on the genetic variants to be mapped.

Referring to FIG. 14, the polygenic risk score, attribute value, and variant contribution score calculation step (S200) may include a step (S210) of calculating a polygenic risk score of genes based on a first algorithm, a step (S220) of calculating a gene contribution score, a step (S230) of calculating an attribute value of genes based on a second algorithm, and a step (S240) of calculating a variant contribution score based on a third algorithm.

The step (S210) of calculating a polygenic risk score of genes based on the first algorithm may be a step of calculating a polygenic risk score based on a preset first algorithm. Here, the preset first algorithm may be in a form of Equation 1, PRS=β×X, where β is a weight vector of genetic variants and X is a genotype matrix.

The step (S220) of calculating the gene contribution score may be a step of calculating the gene contribution score based on the polygenic risk score. Here, the gene contribution score may be calculated based on the equation CS_{(≠i)}=β_{(≠i)} X_{(≠i)}, where β_{(≠i)} is a weight vector of genetic variants included in gene i and X_{(≠i)} is a genotype matrix of genetic variants included in gene i.

The step (S230) of calculating the attribute value of genes based on the second algorithm may be a step of calculating the attribute value based on the polygenic risk score according to a preset second algorithm. Here, the preset second algorithm may be in a form of Equation 3, A_{(≠i)}=CS_{(≠i)}−(CS_{(≠i)})⁻.

The step (S240) of calculating the variant contribution score based on the third algorithm may be a step of calculating the variant contribution score based on a preset third algorithm. Here, the third algorithm may be in a form of Equation 4, CS(snpₖ)=β×x, where β is a weight vector of genetic variants and x is a number of risk genetic variants.

A person having ordinary skill in the art to which the present disclosure pertains will understand that various modifications may be easily made to the present disclosure without departing from the technical spirit or essential features of the present disclosure based on the above description. Therefore, the embodiments described above should be understood as illustrative in all respects and not restrictive. The scope of the present disclosure is defined by the appended claims, and all changes or modified forms derived from the meaning and scope of the claims and their equivalents should be interpreted as being included within the scope of the present disclosure.

### MODE FOR CARRYING OUT THE INVENTION

The mode for carrying out the invention is substantially the same as the best mode for carrying out the invention described above.

### INDUSTRIAL APPLICABILITY

Since the present invention may be used for disease prediction, the present invention has industrial applicability.

What is claimed is:

1. A method performed by a processor, the method comprising:
a) confirming whether input genotype data matches prestored genetic variation risk data and mapping genes and genetic variants based on the genotype data;
b) calculating, based on a plurality of preset algorithms, a polygenic risk score, attributed values, and variant contribution scores for the genetic variants; and
c) providing visualization by visualizing gene contribution scores for each gene at a population level, attributed values for each gene at an individual level, and variant contribution scores for each gene at an individual level based on the attributed values and the variant contribution scores.

2. The method of claim 1, wherein step a) comprises:
a-1) changing a sign of an effect size vector of the genetic variants according to whether the input genotype data matches the prestored genetic variation risk data; and
a-2) mapping the genetic variants to the genes based on first to third methods.

3. The method of claim 2, wherein step a-2) comprises:
performing primary mapping of the genes and the genetic variants based on a distance between the genes and the genetic variants;
performing secondary mapping of the genes and the genetic variants based on relationships between the genes and the genetic variants for which the primary mapping has not been performed; and
extracting genetic variants to be mapped based on relationships between the genetic variants for which the secondary mapping has not been performed and a disease, and performing tertiary mapping by setting a corresponding region within a preset data size based on the extracted genetic variants.

4. The method of claim 1, wherein step a) comprises grouping genes into a single group when genetic variants matching a preset probability or greater correspond through the mapping.

5. The method of claim 1, wherein step b) comprises calculating the polygenic risk score based on a preset first algorithm,
wherein the preset first algorithm has a form of Equation 1, PRS=β×X,
where β is an effect size vector of genetic variants and X is a genotype matrix.

6. The method of claim 5, wherein step b) further comprises:
calculating a gene contribution score based on the polygenic risk score; and
calculating attributed values based on the polygenic risk score according to a preset second algorithm,
wherein the gene contribution score is calculated based on Equation 2, CS_{(≠i)}=P_{(≠i)} X_{(≠i)},
and the preset second algorithm has a form of Equation 3, A_{(≠i)}=CS_{(≠i)}−(CS_{(≠i)})⁻,
where β_{(≠i)} is an effect size vector of genetic variants included in gene i, and X_{(≠i)} is a genotype matrix of genetic variants included in gene i.

7. The method of claim 6, wherein step b) further comprises calculating the variant contribution score based on a preset third algorithm,
wherein the third algorithm has a form of Equation 4, CS(snpₖ)=β×x,
where β is an effect size vector of genetic variants and x is a number of risk genetic variants.

8. A polygenic risk score visualization apparatus comprising:
a communication module;
at least one processor; and
a memory electrically connected to the processor and storing at least one code executed by the processor,
wherein the memory stores code that, when executed by the processor, causes the processor to:
confirm whether input genotype data matches prestored genetic variation risk data,
map genes and genetic variants based on the genotype data,
calculate a polygenic risk score, attributed values, and variant contribution scores for the genetic variants based on a plurality of preset algorithms, and
provide visualization by visualizing gene contribution scores for each gene at a population level, attributed values for each gene at an individual level, and variant contribution scores for each gene at an individual level based on the attributed values and the variant contribution scores.

9. The apparatus of claim 8, wherein the memory stores code that causes the processor to:
change a sign of an effect size vector of the genetic variants according to whether input genotype data matches prestored genetic variation risk data; and
map the genetic variants to the genes based on first to third methods.

10. The apparatus of claim 9, wherein the memory stores code that causes the processor to:
perform primary mapping of the genes and the genetic variants based on a distance between the genes and the genetic variants;
perform secondary mapping of the genes and the genetic variants based on relationships between the genes and the genetic variants for which the primary mapping has not been performed; and
extract genetic variants to be mapped based on relationships between the genetic variants for which the secondary mapping has not been performed and a disease, and perform tertiary mapping by setting a corresponding region within a preset data size based on the extracted genetic variants.

11. The apparatus of claim 8, wherein the memory stores code that causes the processor to group genes into a single group when genetic variants matching a preset probability or greater correspond through the mapping.

12. The apparatus of claim 8, wherein the memory stores code that causes the processor to calculate the polygenic risk score based on a preset first algorithm,
wherein the preset first algorithm has a form of Equation 1, PRS=β×X,
where β is an effect size vector of genetic variants and X is a genotype matrix.

13. The apparatus of claim 12, wherein the memory stores code that causes the processor to:
calculate a gene contribution score based on the polygenic risk score; and
calculate attributed values based on the polygenic risk score according to a preset second algorithm,
wherein the gene contribution score is calculated based on Equation 2, CS_{(≠i)}=β_{(≠i)} X_{(≠i)},
and the preset second algorithm has a form of Equation 3, A_{(≠i)}=CS_{(≠i)}−(CS_{(≠i)})⁻,
where β_{(≠i)} is an effect size vector of genetic variants included in gene i and X_{(≠i)} is a genotype matrix of genetic variants included in gene i.

14. The apparatus of claim 13, wherein the memory stores code that causes the processor to calculate the variant contribution score based on a preset third algorithm,
wherein the third algorithm has a form of Equation 4, CS(snpₖ)=β×x,
where β is an effect size vector of genetic variants and x is a number of risk genetic variants.
